(19) United States
(12) Patent Application Publication
(10) Pub. No.: US 2026/0256788 A1
(43) Pub. Date: Sep. 3, 2026

(54) COMPOSITIONS COMPRISING MELATONIN, 5-FLUOROURACIL AND DIMETHYL SULFOXIDE FOR USE IN THE TREATMENT OF ACTINIC KERATOSIS

(71) Applicant: RepoCeuticals A/S, Hørsholm (DK)

(72) Inventors: Lars Otto Uttenthal, Madrid (ES); Lasse Lindblad, Ålsgårde (DK)

(21) Appl. No.: 19/164,932

(22) PCT Filed: Mar. 12, 2024
(86) PCT No.: PCT/EP2024/056544

§ 371 Date: Sep. 12, 2025

(30) Foreign Application Priority Data
Mar. 14, 2023 (DK) PA 2023 70132

(51) Int. Cl. A61K 31/513; A61K 9/00; A61K 31/4045; A61K 47/20; A61P 17/12

(52) U.S. Cl. CPC A61K 31/513; A61K 9/0014; A61K 31/4045; A61K 47/20; A61P 17/12

(57) ABSTRACT

Compositions are provided that comprise melatonin, 5-fluorouracil, and dimethyl sulfoxide for use in the treatment of actinic keratosis by topical application to the affected skin.

### FIELD OF INVENTION

The present invention provides compositions comprising melatonin, dimethyl sulfoxide and 5-fluorouracil for use in the treatment of actinic keratosis by topical application to a limited area of skin showing multiple lesions of actinic keratosis. As such, it is particularly relevant to the field of dermatology.

### BACKGROUND OF THE INVENTION

### Actinic Keratosis

Actinic keratosis (AK), also known as senile keratosis and solar keratosis, is a common skin disease affecting the epidermis. The disease is regarded as pre-cancerous and typically arises in skin areas that are chronically exposed to sunlight, such as the face, balding scalp, arms and hands. It presents as erythematous, hyperkeratotic, scaly papules in areas of chronically sun-damaged skin and is one of the most common skin disorders evaluated by dermatologists (Hashim et al 2019). The lesions of AK consist of disorganized neoplastic proliferations of epidermal keratinocytes, and as the number of neoplastic cells increases, the lesions become palpable. However, the surrounding epidermis may also contain non-palpable AK lesions as an example of field cancerization, so that strategies for preventing and treating the disease should be directed at the affected area as a whole.

Histologically, the neoplastic keratinocytes are characterized by a disorganized differentiation and orientation, pale or vacuolized eosinophilic cytoplasm, pleomorphic nuclei and an increased number of mitoses. The lesions are classified into three grades of keratinocytic intraepidermal neoplasia (KIN I, II and III) according to whether the cellular atypia occupies the lower third, the lower two thirds or the full thickness of the epidermis.

The lesions may regress spontaneously in approximately one quarter of cases, but may recur in some of these cases, and a variable proportion ranging from 1 in 4000 to approximately one sixth progress to invasive squamous cell carcinoma (SCC), the rest of the cases remaining stable. The overall risk of progression to SCC is approximately 8%, varying with age (higher with advancing age), gender (higher in males), chronic exposure to UV light (higher with higher exposure) and with the location of the lesions. This relates to the risk factors for developing the disease, which are advancing age, male gender, fair skin, geographical proximity to the equator, to which must be added immunosuppression and a previous history of AK or skin malignancies.

AK is usually diagnosed clinically or by use of dermoscopy, but a biopsy can also be taken to rule out SCC or in cases of treatment failure.

Currently, the treatment of AK is either to remove the individual lesions by cryotherapy, curettage, and surgery, or to use field-directed therapies aiming to treat multiple, widespread and subclinical lesions, such as topical medication with 5-fluorouracil (5-FU), imiquimod, or diclofenac), or by photodynamic therapy. However, there are several disadvantaged with the current topical regimens. 5-FU has side effects such as prominent inflammation, ulceration and scarring, and is associated with a lower quality of life for the patients. Imiquimod can induce autoimmune reactions such as alopecia areata and vitiligo. Diclofenac appears to be less effective and has shown poor clearance rates while producing significant irritation. Overall, the current topical agents for AK treatment have significant negative features involving irritation, decreased quality of life and limited efficacy. There is a need to develop topical treatments that target the pathophysiology of AKs without creating skin irritation.

### SUMMARY OF THE INVENTION

The present invention provides dermatological compositions comprising melatonin, 5-fluorouracil (5-FU) and dimethyl sulfoxide (DMSO) for use to improve the treatment of AK by direct topical administration to the affected skin, preferably in the form of a cream or lotion or hydrogel or other semiliquid or liquid dermatological preparation. The DMSO dissolves the melatonin and prevents its breakdown on storage in aqueous media, as well as enhancing the penetration of the melatonin and the 5-fluorouracil into skin cells. The advantage of the present invention is that the melatonin is delivered at high dose directly to the skin that is affected by AK, where the melatonin exerts its own anti-cancerous cellular actions, potentiates the anti-cancerous action of 5-FU, and diminishes the inflammatory and other harmful side effects of 5-FU on the skin.

Accordingly, the present invention provides compositions comprising
a) melatonin,
b) 5-fluorouracil (5-FU) and
c) dimethyl sulfoxide (DMSO)
   for use in the treatment of actinic keratosis (AK), wherein the composition is formulated for topical application to areas of skin affected by AK.

In one embodiment, the composition for use according to the present invention is formulated as a liquid solution, or hydrogel, or lotion, or cream.

In another embodiment, the composition for use according to the present invention contains melatonin at a concentration ranging from 0.1% (w/w) to 15% (w/w), 5-FU at a concentration ranging from 0.1% (w/w) to 5% (w/w), and DMSO at a concentration ranging from 5% (w/w) to 50% (w/w).

In the following detailed description of the invention, details of the scope of the invention will be given, together with details of the practical performance of the invention.

### DETAILED DESCRIPTION OF THE INVENTION

In describing the embodiments of the invention, specific terminology will be resorted to for the sake of clarity. However, the invention is not intended to be limited to the specific terms so selected, and it is understood that each specific term includes all technical equivalents which operate in a similar manner to accomplish a similar purpose.

When describing the embodiments of the present invention, the combinations and permutations of all possible embodiments have not been explicitly described.

Nevertheless, the mere fact that certain measures are recited in mutually different dependent claims or described in different embodiments does not indicate that a combination of these measures cannot be used to advantage. The present invention envisages all possible combinations and permutations of the described embodiments.

The terms “comprising”, “comprise” and “comprises” herein are intended by the inventors to be optionally substitutable with the terms “consisting of”, “consist of” and “consists of”, respectively, in every instance.

As used herein, the singular forms “a”, “an” and “the” are intended to include the plural forms as well, unless the context clearly indicates otherwise.

### Active Ingredients

The active ingredients of the compositions of the invention are melatonin and 5-FU, while DMSO is an important solvent for these ingredients.

Melatonin: Melatonin (N-acetyl-5-methoxytryptamine) is a hormone produced by the pineal gland and enteroendocrine cells of the gastrointestinal tract of human beings and other mammals by the enzymatic modification of the amino acid tryptophan.

Melatonin from the pineal gland is involved in maintaining the circadian rhythm of various biological functions, being secreted in hours of darkness and acting on high-affinity melatonin Gi-coupled transmembrane receptors MT1 and MT2, which are widely distributed in many cells and tissues of the body. At the same time, melatonin acts at supraphysiological concentrations as a powerful antioxidant and free radical scavenger for reactive oxygen species (ROS) and reactive nitrogen species (RNS).

Melatonin can also activate cytoprotective antioxidative enzymes such as copper-zinc and manganese superoxide dismutases (CuZnSOD and MnSOD) and glutathione peroxidase. In addition, melatonin has anti-inflammatory effects to prevent the upregulation or cause the down-regulation of the expression of nuclear factor kappa B (NF-κB) and pro-inflammatory cytokines such as tumor necrosis factor alpha (TNF-α) and interleukin 1 beta (IL-1β).

Melatonin as an antineoplastic agent: Melatonin has been found to exert an antitumor role in several different types of human cancers, including pancreatic, liver, breast and colorectal cancers. It may reduce tumor cell activity in a dose-dependent manner. However, these effects are generally insufficiently potent to be translated into the use of melatonin as a single therapeutic agent for the treatment of cancers. In conjunction with other cancer treatments, such as the use of cytotoxic drugs and radiation, many studies have reported positive effects of melatonin in potentiating the inhibition of tumor cell proliferation and exerting a beneficial effect on survival.

Melatonin affects multiple cellular pathways involved in tumor cell activity and their downregulation. Examples of affected pathways include the following: decreasing the expression of the Murine Double Minute 2 (MDM2 ) protein, which negatively regulates the transcription of the protein p53 and binds to and blocks p53. Melatonin thereby allows p53 to become active. p53 plays a critical role in tumor suppression mainly by inducing growth arrest, apoptosis, and senescence, as well as by blocking angiogenesis. In addition, p53 generally confers cancer cell sensitivity to chemotherapeutic agents and radiotherapy. Likewise, melatonin has a growth inhibiting effect on tumor cells mediated by EGFR suppression and stimulates apoptosis in various tumors by regulating the Bcl-2 and Bax pathway. Moreover, melatonin selectively prevents the signal transduction of metastatic pathways in tumor cells, and inhibits metastasis and tumor invasion. It has been shown to inhibit the JNK/MAPK pathway in lung cancer cell lines, which among other pathways is activated by RAS, and the PI3K/Akt pathway in breast and esophageal cancer cell lines. It also triggers the release of cytochrome C and stimulates caspase activity resulting in the activation of the intrinsic apoptosis pathway. Further examples of pathways affected by melatonin are given below. These pathways are involved in the complex pathophysiology of AK and the associated field cancerization, as well as in SCC and the transition to SCC.

Topically applied melatonin can therefore alleviate these pathological changes associated with AK.

Melatonin as an anti-inflammatory agent: Anti-inflammatory actions of melatonin that are relevant to the inflammatory component in AK include the inhibition of COX-2 expression and prostaglandin E production. Melatonin also downregulates NF-κB, which promotes the activity of COX-2, and TNF-α, resulting in a reduced overproduction of leukocytes and pro-inflammatory cytokines.

5-FU: 5-FU (5-fluorouracil or 5-fluoro-2,4(1H;3H)-pyrimidinedione, often simply called fluorouracil) is a pyrimidine analogue that inhibits the enzyme thymidylate synthase via its metabolite fluorodeoxyuridine monophosphate (FdUMP), thus blocking the synthesis of DNA. This disrupts cell division, affecting more rapidly dividing neoplastic cells than slower-dividing normal cells and forms the basis for its use as a cytotoxic cancer therapeutic agent in a variety of cancer types, typically showing a low therapeutic index and causing notable toxic side effects. The triphosphate metabolite FdUTP can affect cell metabolism in various ways, such as interfering with transcription, translation and post-translational modification through incorrect addition into RNA. Topically applied 5-FU is currently the most effective treatment for AK. However, it has serious adverse effects, its use being associated with skin inflammation, erosion and ulceration during treatment and for one or two weeks after treatment has ended.

Cooperative effect of melatonin and 5-FU: Melatonin potentiates the antineoplastic effects of 5-FU in the treatment of actinic keratosis by a cooperative or even synergistic action on several pathways. It is here proposed that one of these synergistic actions may be on the expression of the histone-lysine N-methyltransferase enzyme, enhancer of Zeste Homolog 2 (EZH2), which is increased in the neoplastic keratinocytes of AK and even more on transition to SCC. Melatonin may sensitize the cells to the inhibitory effect of 5-FU on EZH2 expression and thereby impair the effect of EZH2 to activate the JAK2/STAT3 signaling pathway involved in tumorigenesis. EZH2 also contributes to tumor cell resistance to 5-FU, and this resistance may be impaired by the action of melatonin. A result of the combined action of melatonin and 5-FU is an increased apoptosis of tumor cells via the Bcl-2/Mcl-1/cleaved caspase-3 pathway.

The use of 5-FU alone to treat cancers may induce tumor cell resistance to 5-FU through an increase in phosphorylated ERK (pERK) and phosphorylated Akt (pAkt). Melatonin inhibits the phosphorylation of ERK and Akt, abrogating the resistance of the cells to 5-FU and resulting in a synergistic effect with 5-FU on tumor cell viability. Melatonin also acts synergistically with 5-FU by suppressing the PI3K/Akt and NF-κB/iNOS signaling pathways.

Amelioration by melatonin of the adverse effects of 5-FU: Melatonin is well attested to diminish the adverse effects of many cytotoxic drugs used to treat cancers, including 5-FU, while not diminishing the efficacy of these drugs against the cancer. In the present compositions, the anti-inflammatory action of melatonin is particularly relevant to suppressing the inflammatory effects of 5-FU.

DMSO as a solvent for melatonin and 5-FU: DMSO, dimethyl sulfoxide (chemical formula (CH₃)₂SO, molecular weight 78.1 g/mol), is a colorless polar aprotic solvent for both polar and nonpolar compounds and is completely miscible with water and a wide range of organic solvents. It is well known as a solvent or solubilizer for melatonin. The solubility of melatonin in DMSO at room temperature may be as high as 232 g/L or higher. 5-FU is more polar than melatonin, but is only sparingly soluble in water, while very soluble in DMSO. DMSO shows low toxicity, the median lethal dose being higher than that of ethanol (DMSO: LD₅₀ oral, rat, 14.5 g/kg; ethanol: LD₅₀ oral, rat, 7.06 g/kg). DMSO penetrates the skin and other epithelia without damaging them and can carry other compounds dissolved in it into the underlying cells. In relation to the treatment of AK, DMSO aids the penetration of melatonin and 5-FU through the stratum corneum and into the underlying cells of the epidermis, including the neoplastic keratinocytes. DMSO has been used in human subjects as a topical analgesic, a vehicle for the topical application of pharmaceuticals e.g. as a component of a transdermal drug delivery systems, as an anti-inflammatory agent, and as an antioxidant. In the compositions of the present invention, DMSO not only acts as a solvent and penetration enhancer of the active ingredients but also as a stabilizer of melatonin in aqueous media, prolonging the shelf-life of the compositions.

### Formulations

The compositions of the present invention may be formulated in any way that make them suitable for delimited topical application to an area of skin affected by AK, such formulations comprising but not necessarily being limited to a liquid solution, or hydrogel, or lotion, or cream. The present composition ensures the melatonin is delivered at high dose directly to the skin that is affected by AK.

The formulation typically contains melatonin at a concentration ranging from 0.1% (w/w) to 15% (w/w), i.e. from 1 mg to 150 mg of melatonin per gram of formulated composition; 5-FU at a concentration ranging from 0.1% (w/w) to 5% (w/w), i.e. from 1 mg to 50 mg of 5-FU per gram of formulated composition; and DMSO at a concentration ranging from 5% (w/w) to 50% (w/w), i.e. from 50 mg to 500 mg per gram of formulated composition. In any such formulation, the weight of DMSO present per gram of formulation will always exceed the combined weight of melatonin and 5-FU in a gram of the formulation.

In a preferred embodiment, the formulation is made up as a lotion or soft, readily spreadable cream, using any suitable basic dermatological lotion or cream formulation known in the art as a basis for the final formulated composition, provided that all the excipients of the formulation are chemically compatible with the active ingredients.

In one embodiment of the formulation, melatonin is present at a concentration ranging from 0.1% (w/w) to 15% (w/w), such as from 0.5% (w/w) to 12.5% (w/w), or from 1% (w/w) to 10% (w/w), e.g. from 2% (w/w) to 7.5% (w/w), or from 2.5% (w/w) to 5% (w/w). For example, melatonin may be present at a concentration ranging from 12.5% (w/w) to 15% (w/w).

In another embodiment of the formulation, 5-FU is present at a concentration ranging from 0.1% (w/w) to 5% (w/w), such as from 0.2% (w/w) to 3% (w/w), e.g. from 0.3% (w/w) to 2% (w/w), or from 0.5% to 1% (w/w). For example, 5-FU may be present at a concentration ranging from 4% (w/w) to 5% (w/w).

In another embodiment of the formulation, DMSO is present at a concentration ranging from 5% (w/w) to 50% (w/w), such as from 10% (w/w) to 25 % (w/w), e.g. from 10% (w/w) to 15 % (w/w). For example, DMSO may be present at a concentration ranging from 40% (w/w) to 50% (w/w).

An embodiment of the above formulation comprises melatonin 2.5% (w/w), 5-FU 0.5% (w/w) and DMSO 15% (w/w) formulated as a cream.

Another embodiment of the above formulation comprises melatonin 2.5% (w/w), 5-FU 4% (w/w) and DMSO 15% (w/w) formulated as a cream.

Another embodiment of the formulation comprises melatonin 15% (w/w), 5-FU 5% (w/w) and DMSO 50% (w/w) formulated as a cream.

Yet another embodiment of the formulation comprises melatonin 15% (w/w), 5-FU 4% (w/w) and DMSO 50% (w/w) formulated as a cream.

Solutes that may be added to the water in the formulated composition include pH-adjusting agents such as hydrochloric acid, sodium hydroxide and biocompatible buffering agents, non-limiting examples being sodium dihydrogen phosphate and disodium hydrogen phosphate, sodium carbonate and bicarbonate. Tonicity-adjusting agents, such as for example sodium chloride or calcium chloride, may also be added, as well as suitable preservative agents such as methyl and/or propyl parahydroxybenzoate.

### Administration

Administration of the composition is by topical application to a continuous area of skin that is affected by several AK lesions and has been defined by the treating clinician as being an area that is suitable for topical treatment. A hydrogel, cream or lotion formulation may be applied thinly with an applicator or with the fingers, preferably protected by a thin vinyl, nitrile or latex glove. If the fingers are in direct contact with the preparation, the hands should be washed immediately after the application. A liquid formulation may be applied with a small brush. The formulation is applied thinly and evenly to the skin area to be treated. The clinician may determine that the maximum area of skin to be treated at any one time is limited to 100 square centimeters or even to 25 square centimeters. In an embodiment, the formulation is administered to the lesion and the closest adjacent area of the lesion up to 1 cm from the lesion border.

The compositions are intended to be applied to the designated skin area once daily, immediately before retiring. The duration of treatment may be for 1, 2, 4, 8 or 12 weeks, depending on the clinician's assessment of the response to treatment.

### EXAMPLE

### Clinical Trial to Determine the Effect of a Composition of this Invention Administered Topically to the Skin of Patients with Actinic Keratosis

Design: A clinical phase II, randomized, controlled, open-label trial investigating the effect of 5-fluorouracil, melatonin, and a combination of melatonin and 5-fluorouracil.

Inclusion: Participants with a clinical diagnosis of actinic keratosis, 18 or more years of age, who have signed an informed consent.

Exclusion: Known allergic reaction to an active ingredient, on immunosuppressive therapy, and women of childbearing age.

Intervention: Patients are randomized into 4 groups, each of 12 patients on completion of the trial.

Group 1 receives daily treatment with a 4% w/w 5-fluorouracil cream for 10 days.

Group 2 receives daily treatment with a cream containing 2.5% w/w melatonin, and 15% w/w DMSO for 10 days,

Group 3 receives daily treatment with a cream containing 4% w/w 5-fluorouracil, 2.5% w/w melatonin, and 15% w/w DMSO for 10 days.

Group 4 receives no treatment, but a punch skin biopsy is taken from an actinic keratosis lesion and another from normal buttock skin before the patient is started on a standard treatment for actinic keratosis.

A single punch biopsy from a treated lesion is taken on day 14 from each patient in groups 1, 2 and 3.

Clinical assessment, photographs and colorimetric measurements of the lesions in patients of groups 1, 2 and 3 are made on the day of inclusion and on days 5, 10 and 14.

### Outcome Measures

Clinical, photographic and colorimetric assessment of the effects of treatment.

Assessment of formalin-fixed and paraffin-embedded sections of the punch skin biopsies for histopathological changes by conventional staining, inflammatory markers by immunohistochemical staining, and for changes in gene and protein expression using commercially available kits for this purpose, such as those supplied by NanoString Technologies (Seattle, WA, USA).

### REFERENCE

Hashim PW, Chen T, Rigel D, Bhatia N, Kircik LH (2019) Actinic keratosis: current therapies and insights into new treatments. J Drugs Dermatol. 2019 May 1;18(5 Suppl 1):s161-166.

1. A method for treating actinic keratosis (AK) in a subject in need thereof comprising:
administering a composition formulated for topical application to areas of skin affected by AK, wherein the composition comprises:
a) melatonin,
b) 5-fluorouracil (5-FU), and
c) dimethyl sulfoxide (DMSO).

2-8. (canceled)

9. The method of claim 1, wherein the composition is formulated as a liquid solution, hydrogel, lotion, or cream.

10. The method of claim 1, wherein the melatonin is present at a concentration ranging from 0.1% (w/w) to 15% (w/w) or wherein the melatonin is present at a concentration ranging from 12.5 % (w/w) to 15% (w/w).

11. The method of claim 1, wherein the 5-FU is present at a concentration ranging from 0.1% (w/w) to 5% (w/w) or wherein the 5-FU is present at a concentration ranging from 4% (w/w) to 5% (w/w).

12. The method of claim 1, wherein the DMSO is present at a concentration ranging from 5% (w/w) to 50% (w/w) or wherein the DMSO is present at a concentration ranging from 40% (w/w) to 50% (w/w).

13. The method of claim 1, wherein the concentration of melatonin is 2.5% (w/w), the concentration of 5-FU is 0.5% (w/w), the concentration of DMSO is 15% (w/w), and the composition is formulated as a cream.

14. The method of claim 1, wherein the concentration of melatonin is 2.5% (w/w), the concentration of 5-FU is 4% (w/w), the concentration of DMSO is 15% (w/w), and the composition is formulated as a cream.

15. The method of claim 1, wherein the concentration of melatonin is 15% (w/w), the concentration of 5-FU is 5% (w/w), the concentration of DMSO is 50% (w/w), and the composition is formulated as a cream.
